(19) United States
(12) Patent Application Publication
(10) Pub. No.: US 2009/0186554 A1
(43) Pub. Date: Jul. 23, 2009

(54) Pad printing with vitreous enamels

(75) Inventor: Antonio R. Alfaro, Upper Black Eddy, PA (US)

Correspondence Address: DILWORTH PAXSON LLP, 1500 Market Street, Suite 3500 E, PHILADELPHIA, PA, 19102, US

(73) Assignee: Tohickon Corporation, Ottsville, PA (US)

(21) Appl. No.: 12/321,511

(22) Filed: Jan. 21, 2009

Related U.S. Application Data
(60) Provisional application No. 61/011,681, filed on Jan. 18, 2008.

(51) Int. Cl. A63H 33/00; C09D 11/10; A61L 27/16

(52) U.S. Cl. 446/389; 523/160; 427/2.24

(57) ABSTRACT

An improved ink composition and method for printing such inks on smooth, flat, curved uneven and/or irregular surfaces and substrates is disclosed. The ink may comprise a vitreous enamel in dry powder form; a clear vinyl based ink; and a thinner, wherein the ink is able to be utilized in the printing of images on glass, glass-like, ceramic, metal, metal-like, or other such substrates. The inks and method of printing the inks may be used in the manufacture of artificial eyes or other substrates. Such artificial eyes may comprise a glass, glass-like, ceramic, or other such substrate, and graphical or decorative inks which are bonded to the substrate. Generally, the decorative ink is applied to the inner or convex surface of the substrate, preferably by pad printing techniques and subjected to heat which bonds the ink to the surface of the substrate. The decorative inks are highly resistant to physical wear, separation from the substrate, chemical reaction, and other degradation. Thus, the inks of this invention do not require the use of a protective or barrier layer or other coating to achieve such resistance and durability.

### CROSS-REFERENCE TO RELATED APPLICATIONS

This application claims the priority of U.S. Provisional Patent Application Ser. No. 61/011,681, filed Jan. 18, 2008, entitled “Pad Printing with Vitreous Enamels” the entirety of which is incorporated herein by reference.

### FIELD OF INVENTION

This invention is directed to inks which may be utilized in printing on smooth, flat, curved and/or uneven surfaces. More specifically, this invention is directed to inks which may be applied to substrates, and particularly substrates having surfaces which are not conducive to inks adhering to such surfaces, such as for example, glass and glass-like surfaces used in the production of artificial eyes, as well as ceramics, metal and metal-like surfaces.

### BACKGROUND OF THE INVENTION

Printing inks on smooth, flat, curved and/or uneven surfaces such as glass, glass-like, ceramic, metal, metal-like and other such substrates, and permanently adhering inks to such surfaces has long been a problem encountered in the printing arts. In this regard, inks do not readily adhere or remain affixed to such surfaces. In addition, the inks are prone to physical wear, scratching, shrinkage and separation from the substrate, chemical reaction to materials with which the ink comes in contact and other forms of degradation.

At present, artificial eyes, and particularly artificial eyes of high quality used in taxidermy, are hand painted by highly skilled artisans. These high quality artificial eyes are often made of glass, crystal or other glass-like substrates which are typically decorated with paints or inks comprising fritted enamels. Once applied to the substrate, the inks are fired into the glass using ovens operating at high temperatures. The firing process causes the enamels to become affixed to the glass substrate, and bonds the enamel to the glass creating a very durable combination of enamel to substrate.

Although effective, the manual application of these enamels is labor intensive, and thus time consuming and costly. Unfortunately, enamels used on glass-like substrates are not conducive to automated application methods. Upon initial consideration, established pad printing techniques might appear to be a suitable choice in automating the process of applying inks to glass-like substrates. However, as explained below, pad printing techniques, are not easily adapted to the automated printing of inks on glass. Before addressing such problems, however, a brief description of the general principals of pad printing is warranted.

Pad printing is a method of printing designs or text onto flat, curved, or uneven surfaces. Pad printing may be accomplished with a semi-automatic or fully automated machine. See, for example, the Printex, Inc. pad printer shown at A-49, A-56, and A-57 of Appendix A attached hereto. First a photopolymer or steel plate is etched with the design to be transferred creating a series of depressions in an otherwise flat plate in order to hold the ink to be transferred in the pattern of the design. A cup filled with a pad printing ink is placed over this design. This cup has a flat edge which contacts the plate evenly so that when the cup is moved laterally across the plate, a squeegee effect is created, keeping all the ink in the cup save that which was deposited in the valleys and depressions etched into the plate. A silicone pad is then positioned over the image and pressed down upon it. The ink is engineered to stick to the pad and stay in the shape of the design. The same pad is then positioned over the substrate one wishes to print and then is pressed down upon it. The ink then releases from the pad onto the substrate. These specially engineered inks, the machines, and the silicone pads are available to the public and generally known to the printing industry. Typically, vinyl acrylic based inks are used in pad printing applications, however such inks do not permanently bond to glass, ceramic or other glass-like substrates.

Though pad printing inks are specially engineered to adhere to a silicone pad only well enough to travel to a substrate and have a tendency to transfer to any substrate other than silicone more readily, printing on glass tends to be problematic. Glass in an extremely smooth surface, thereby offering little or no surface area to which the ink may be affixed. By contrast, however, substrates such as plastic or paper, for example, provide a more textured surface to which inks may adhere.

Pad printing techniques have been employed with some success in the production of a plastic eyes used in taxidermy. In this regard, it is believed that pad printing has been used in the production of plastic artificial deer eyes. With plastic substrates, adhesion of the ink is more easily achieved and no catalyst or baking is necessary. Though printing on plastic substrates may avoid many of the problems of printing on glass substrates, plastic has other significant drawbacks as a substrate particularly with when used in the manufacture of artificial eyes. Perhaps most significant of such drawbacks is that plastic eyes are not very durable. Specifically, the plastic itself is easily scratched, harmed by chemicals, and prone to problems with clarity due to inherent imperfections in the plastic substrate. In contrast, for many applications, glass substrates are far superior to plastic inasmuch as glass remains clear and does not cloud, does not easily scratch, and is highly resistant to chemicals.

Although glass is a preferred substrate in many applications, known pad printing techniques are not well suited to the automated application of inks to glass-like surfaces. In this regard, the inks do not adhere well to smooth glass-like surfaces. The inks are susceptible to: (1) physical wear and scratching; (2) chemical reactions to other materials with which the ink may come in contact; and (3) other forms of degradation. For example, cleaning solvents and acidic based materials, such as mounting clays used in taxidermy to mount artificial eyes to taxidermy manikins, tend to weaken the already weak bonds between the inks and glass-like substrate.

Even when the pad printing inks are mixed with a glass hardening catalyst and heat cured, the inks may not sufficiently adhere to the inside surface of the glass eye. Accordingly, when epoxies or other adhesives are used to affix the glass eye to a manikin or other form, the epoxies or other adhesives tend to shrink causing the inks to “delaminate” from the glass substrate inasmuch as the bond between the ink and epoxy is stronger than the bond between the ink and the glass. In addition, where acidic mounting clays are used in mounting the eye to a manikin or other form, such clays may come in contact with the inks. In many instances, the inks are not able to withstand the acids which may leach from the clays. Furthermore, a taxidermist may use lacquer thinner or acetone during the mounting process to clean unwanted clay or paint from the outside of the eye. Use of such solvents may seep to the inside of the eye causing degradation of the printed inks.

Thus, artificial glass eyes are exposed to more rigorous treatment and environments than other painted or printed products. Artificial glass eyes may be used in a number of applications such as doll making, sculpture, and taxidermy, a particularly harsh application for glass eyes.

For example, in the process of mounting artificial eyes in taxidermy applications mentioned above, a taxidermist typically sets a glass eye into a form or taxidermy manikin by filling the inside of its hemispherical shape with clay or a two part epoxy. The same clay or epoxy is also used to sculpt an anatomically correct eyelid area over the eye for the animal hide to lay over the eye in a natural way. A layer of hide paste is then applied to the surface of the manikin. The hide of the animal is then pulled over the form with eye set in, positioned properly, seems are sewn and it is left to dry. This is the bulk of the labor involved in most taxidermy and once it is left to dry, any adjustments to eye set are very difficult and more labor intensive than the original setting of the eye.

In the instance of the production of an artificial eye using a pad printer and the specialized inks for the process described herein, the inks printed on the glass substrate may not withstand the rigors of the taxidermy eye mounting process described above. For example, certain mounting clays have a mild acidity which would attack the inks causing them to dissolve and discolor. This is particularly a problem when the clay is first applied and is drying. If a taxidermist inadvertently uses acidic clay, the acids may discolor the printed eye. Even though many taxidermists utilize pH neutral (not acidic or basic) clay, some batches of such clays contain trace acidity which may contain enough acid to adversely affect the inks.

In addition, epoxies are also widely used in the eye mounting process. The epoxies, which are applied to and/or come in contact with the inks, tend to shrink as the epoxy sets. The shrinkage of the epoxy puts a tremendous amount of pressure on the surface of the decorative ink layer and, in at least some cases, the ink may delaminate from the concave surface of the glass substrate. In severe cases, the glass eye may fracture as a result of epoxy shrinkage.

One option may be to apply a hard coating adhered to the ink layer with an epoxy or other adhesive. However, hard coatings have a tendency to aid in delamination by spreading the pressure caused by epoxy shrinkage over the inside surface of the glass eye substrate thereby pulling the ink off of the glass surface.

Another option may be to apply a hard backing, such as for example, a flat plastic disk affixed to the back or the concave surface of the glass eye substrate. However, the fabrication and application of such a disk would prove difficult and costly. In one embodiment, the disk would be adhered to the edge of the glass eye, which is often a thin edge, thereby providing little surface area for adhesion. Thus, the disk may detach from the edge of the glass eye, or not be affixed properly, leaving the ink layer exposed to harmful effects and agents.

Another option, described in U.S. patent application Ser. No. 11/891,942, filed Aug. 13, 2007, the entirety of which is incorporated herein by reference, may be to apply a barrier layer over the decorative inks. The barrier layer may be comprised of almost any rubberized or other flexible and/or protective coating suitable to the particular application. For example, the barrier layer may comprise silicone rubber, urethane rubber, butyl rubber, ethylene propylene diene monomer rubber, fluoroelastomers, rubberized automotive undercoating, sprayable mold releases, sprayable waxes, wax mold release materials, epoxy powder coatings, or the like.

However, at present, no inks are available which are suitable for use in the automated pad printing of glass or glass-like substrates where the inks are resistant to physical wear and/or chemical degradation. Accordingly, there exists a need for inks utilized in the pad printing of glass and glass-like substrates that: (1) produce a substantially anatomically correct likeness of an eye found in nature is produced by the printing process; (2) are consistently and repetitively applied to multiple artificial glass eyes; (3) are easily applied to and substantially completely release from ink application devices; (4) substantially completely and permanently adhere to glass-like substrates; (5) resist shrinkage, separation, wear, scratching, and/or reaction with other materials.

Thus, there exists a need for inks utilized in the pad printing of glass and glass-like substrates which comprise improved characteristics of adhesion to glass and glass-like substrates, and are more durable when used in taxidermy and other applications.

### SUMMARY OF THE INVENTION

The difficulties encountered in applying and adhering printing inks to glass substrates may be, in part, overcome by using specially formulated inks with a specially adapted pad printing machine. In this regard, specially formulated vitreous enamels of this invention may be employed.

Vitreous enamels are powder based pigments that are generally mixed with a fluid medium for application to glass, ceramics and/or metals. Generally, vitreous enamels contain elements that allow the pigment to permanently adhere to the substrate by firing at high temperature.

The application of vitreous enamels to glass and ceramic substrates has been traditionally achieved by hand painting, spraying, and through the use of decalcomania transfers. All of these methods require skilled artisans and/or complicated and expensive automation.

Application by hand is prone to inconsistency by human error. Decalcomania transfers, though generally consistent in design, are often applied by hand and can be misaligned or misplaced. Furthermore, they are prone to failure if they are not properly applied or if they are allowed to dry out prior to application.

Conventional pad printing inks are generally a vinyl or acrylic base and cannot be permanently bonded to glass or ceramic. They also cannot be exposed to excessive heat.

Pad printing is a very attractive method of applying vitreous enamels because it enables the consistent application of a design to a glass or ceramic substrate and produces a combination which can then be fired to permanently adhere the enamel to the substrate.

Vitreous enamels are usually mixed with an oil based medium and dispersed in that medium through the use of specialized milling and dispersion equipment. These oil based mediums are usually thixotropic in nature and thus very sticky. This allows them to stay in place when applied to a smooth, non-porous surface such as glass or ceramic over glaze. When fired the organic materials in the medium first burn off and then when the vitreous enamel reaches its maturing temperature it begins to flow into the surface of the substrate.

However, the stickiness of this medium also presents a problem when pad printing vitreous enamels. Generally the vitreous enamel does not release well from a silicone printing pad. Some transfer does occur but not enough to obtain a quality print. Thus, pad printing vitreous enamels to date has been impractical.

The inks of the present invention may employ mediums from industries outside of the glass and ceramic decorating arena. In one embodiment, specially formulated vitreous enamels may be utilized to successfully and efficiently pad print glass, glass-like, ceramic, metal, and/or metal-like substrates. For example, the ink of this invention may comprise a vitreous enamel ink in powder form mixed with a clear vinyl base and thinned with a suitable thinner.

### BRIEF DESCRIPTION OF THE DRAWINGS

Understanding of the present invention will be facilitated by consideration of the following detailed description of the embodiments of the present invention taken in conjunction with the accompanying drawings, in which like numerals refer to like parts, and wherein:

FIG. 1 is a plan view of the top, convex surface of an embodiment of the artificial eye of the instant invention;

FIG. 2 is a plan view, opposite that of FIG. 1, of the concave surface of the artificial eye of FIG. 1;

FIG. 3 is an elevation of the artificial eye of FIG. 1; and

FIG. 4 is a cross section of the artificial eye of FIG. 1 taken through 4-4 of FIGS. 1 and 2.

FIG. 5A and FIG. 5B combined is a flow diagram of an embodiment of the method of the instant invention.

Additional documents attached hereto constitute additional teachings included in the present invention, including Appendix A hereto comprising photographs which depict an embodiment of the instant invention, and which are incorporated by reference in there entireties herewith.

### DETAILED DESCRIPTION OF THE EMBODIMENTS

It is to be understood that the figures and descriptions of the present invention have been simplified to illustrate elements that are relevant for a clear understanding of the present invention, while eliminating, for the purposes of clarity, many other elements which may be found in the present invention. Those of ordinary skill in the pertinent art will recognize that other elements are desirable and/or required in order to implement the present invention. However, because such elements are well known in the art, and because such elements do not facilitate a better understanding of the present invention, a discussion of such elements is not provided herein.

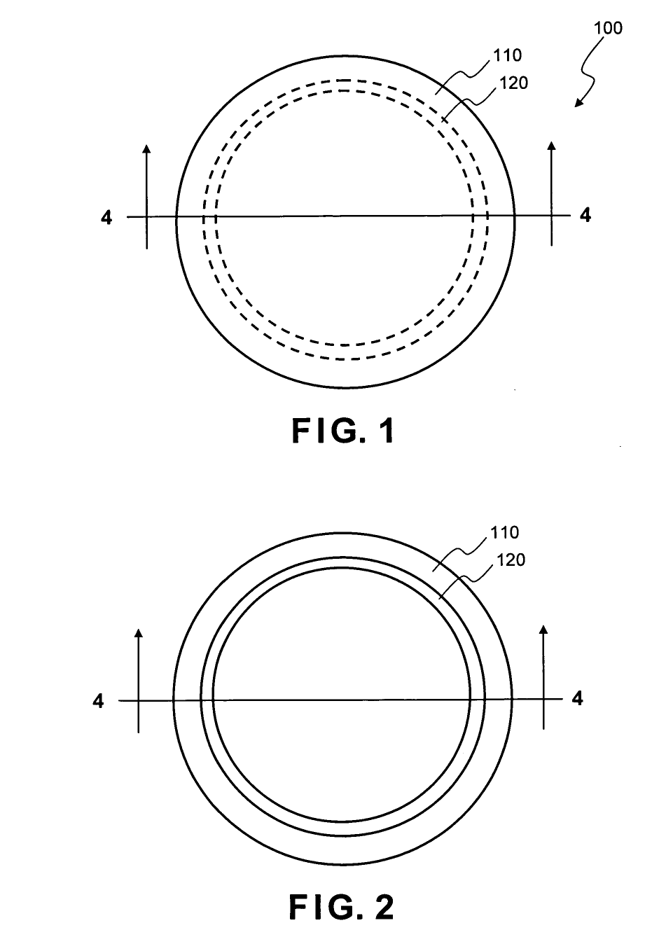

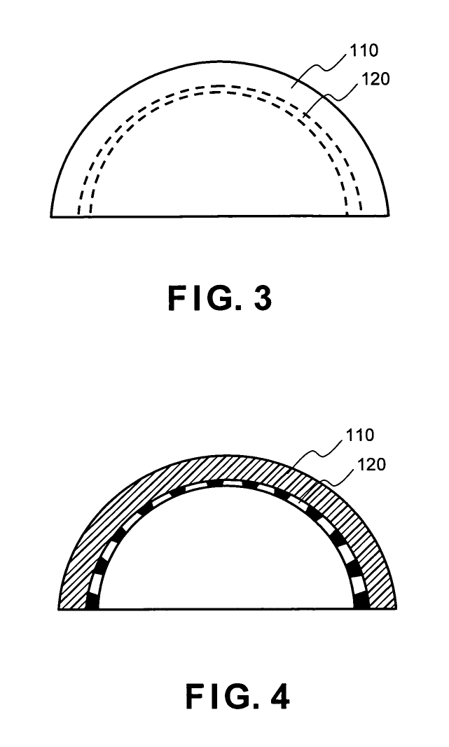

Turning now to FIGS. 1 through 4, there is shown artificial eye 100. Eye 100 comprises substrate 110, graphical or decorative ink 120. Generally, decorative ink 120 is applied to the inner or convex surface of substrate 110. Decorative ink 120 may be applied to substrate 110 in any appropriate manner such as, for example, by employing pad printing techniques or by spraying.

Generally, ink 120 may be produced by mixing a vitreous enamel in dry powder pigment form with an appropriate medium, and then by introducing a thinner, oil or other component to create an ink paste. The vitreous enamel dry powder pigment may comprise about 15% by weight of pigment to about 85% by weight of glass flux, and the medium may comprise a clear dry powder enamel base medium. As described herein, ink 120 may comprise any mixture which produces an ink with the desired viscosity, release characteristics and opacity. In one embodiment, ink 120 may comprise about 50% by volume of vitreous enamel dry powder, about 50% by volume of medium, and between about 10% to about 35% of thinner or oil by weight of the sum of vitreous enamel and medium. Once mixed into an ink paste, ink 120 is milled in a milling machine to crush and disperse the glass flux and other components throughout the ink 120 mixture.

In another embodiment of the instant invention, ink 120 may be mixed with a clear base as the medium in order to lighten the opacity of the color of ink 120 being used in order to make it more translucent. The clear base utilized may be from a line of vinyl based colors from Visprox. This clear base (TCP 9959 Clear Base) is designed for mixing with the Visprox 9900 series inks.

When mixed with vitreous enamels in powder form and thinned using the Visprox thinner from the same Visprox 9900 series line, ink 120 functions very well as a pad printing ink. When pad printing, such a mixture transfers well from the image plate to the printing pad, and completely or substantially completely releases from the printing pad to a glass or glass like substrate.

Varying degrees of opacity in ink 120, as ink 120 appears on substrate 110, may be achieved by printing the same image on the substrate 110 multiple times to obtain desired opacity and/or by varying the pigment load while mixing ink 120 prior to printing. In one embodiment, ink 120 of the present invention comprises: (1) about 50% by volume vitreous enamel in dry powder form; (2) about 50% by volume Visprox Clear Base; and (3) about 30% Visprox 9959 Thinner by weight of the sum of vitreous enamel and clear base mixture. This ink 120 mixture produces a good quality translucent print. Printing the same image multiple times over the same substrate 110 with this ink 120 mixture increases the opacity of the overall image printed.

In another embodiment, the ink of the present invention the ink comprises: (1) about 75% by volume of vitreous enamel in dry powder form; (2) about 25% by volume of Visprox clear base; and (3) and about 30% of Visprox 9959 Thinner by weight of the sum of vitreous enamel and clear base mixture. In this embodiment, an increase in opacity in the printed image was observed over the opacity of the image printed in the first embodiment described above. Also with this embodiment, multiple prints of an image on a single substrate 110 produces an increase in the opacity of the final printed image.

In producing other embodiments of ink 120, the ratio of enamel to clear base to thinner may be adjusted to suit desired ink characteristics in, for example viscosity, release characteristics and opacity.

In yet another embodiment of the invention, the viscosity of ink 120 may be increased by using less thinner. The viscosities of the inks described in the previous embodiments set forth above are relatively thin. About thirty percent (30%) by weight thinner to ink ratio is a suggested starting point for pad printing inks, however, other amounts may be employed depending on the ink characteristics desired.

Generally, vitreous enamels are of a much higher density than conventional pad printing inks. Also the clear base employed in these embodiments is relatively thin in comparison to conventional, pigmented, pad printing inks. Therefore, the thinner utilized in the inks of these embodiments generally has a greater effect on the viscosity the inks per volume. In this regard, it has been observed that inks 120 which contain less thinner produce a more opaque print.

The ink 120 viscosity required for sufficient printing depends, at least in part, upon the quality of the image plate and the type of artwork that is being printed. Under many circumstances, it is desirable that ink mixture 120 is thin enough to permit the ink to be squeegeed or otherwise applied well into the etched recesses of the image plate of a pad printer. See e.g. A-50 through A-55 and A-58 through A-67. Solid art such as lines, text, and solid shapes require a more viscous mixture than halftone art. Halftone art requires a less viscous mixture as it contains finer details on the image plate which the mixture must be able to flow into.

Thus, the desired ink viscosity may be determined by at least: (1) the characteristics of the composition of the ink; (2) the characteristics of the pad printing image plate; (3) the type of artwork being printed (e.g. solid or half-tone images); (4) the characteristics of the material the pad is made from; (5) the shape of the pad; and (6) the pressure applied to the pad.

Another way of approaching the problem of appropriate opacity in the vitreous inks being printed is to create a translucent print and back up the translucent print with an opaque color. This opaque color may be brushed or sprayed on very quickly as it is a general background coating. If one is printing four-color separated art or artwork that encompasses the entire surface of the substrate or decorated area of the substrate, a white coating may be applied to opacify the print and bring trueness to the colors. This is the same principle by which inks are applied to white paper such as with an inkjet printer. One may alternatively use a colored background to alter or enhance the printed colors.

In one embodiment, a background coating may first be printed on a clear glass substrate if the artwork being printed is to be viewed from the same side it is printed on. If the artwork is to be viewed through the glass substrate, then the background coating may be applied last. On ceramic or on opaque glasses the background coating may be applied first or the opaque substrate may substitute for the background coating (e.g. ceramic tile with white or other colored over-glaze, opaque glass, and the like).

It should also be noted that other mediums other than a clear base may work as well as the medium used in the embodiments disclosed herein.

The decoration of realistic glass eyes for any purpose often requires multiple steps in preparing the artwork and printing the inks. The number of steps often depends upon the eye being replicated and the degree of quality, detail and accuracy desired. The method of printing inks 120 of this invention may be carried out via the use of manual, semi-automatic or automatic pad printing device. Such pad printers, which are adaptable to be retrofitted as described herein for the purposes of printing inks 120 of this invention, are available through Printex of Poway, Calif. See www.printexusa.com. Such printers may comprise single or multiple printer heads and/or single or multiple substrate mounting fixtures. One such device is depicted in A-56 through A-71 of Appendix A. This embodiment of the printer comprises two pad printing heads and a double mounting fixture which holds two glass eye substrates 110 (shown in A-56 through A-71 of Appendix A with only one print head and single mounting fixture mounted). The mounting fixture may shuttle laterally between each printing head.

In another embodiment, a Printex printer may be configured with multiple printing stations each of which comprise at least one pad printing head, at least one image plate bearing the design to be printed on substrate 110 and an ink applicator device which applies ink 120 to the image plate. Each printing station is equipped to apply at least one design in at least one color to substrate 110. Substrate 110 is mounted to a mounting fixture and held in a substantially fixed position by a vacuum and/or fixture in the form of substrate 110. Multiple fixtures may then be incorporated into an indexing table, such as a rotary indexing table. The indexing table may then automatically or semi-automatically move the fixtures bearing substrates 110 into position in each printing station so as to permit precision printing of the design on each station's image plate to substrate 110. In this regard, this configuration permits the same design to be printed multiple times on a single substrate 110 in order to, for example, increase the opacity of the color of the design being printed on substrate 110.

This configuration substantially eliminates the problems associated with batch process systems where substrate 110 must be moved by hand from printing station the printing station and realigned relative to the printing pad which bears the design to be printed. In this embodiment remains fixed in the same mounting fixture and in the same orientation between the printing pad of each printing station as the mounting fixture bearing substrate 110 is indexed from printing station t printing station. Thus remounting and reorientation of substrate 110 are avoided.

In one embodiment of the Printex printer, the printer comprises five separate print stations and a rotary indexing table which comprises eighteen mounting fixtures. In this embodiment, up to five stations may be utilized at one time with each station printing a different color and/or design.

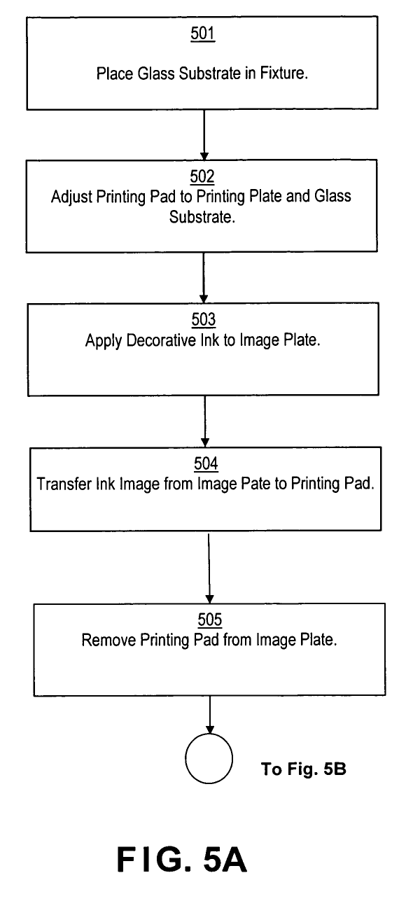

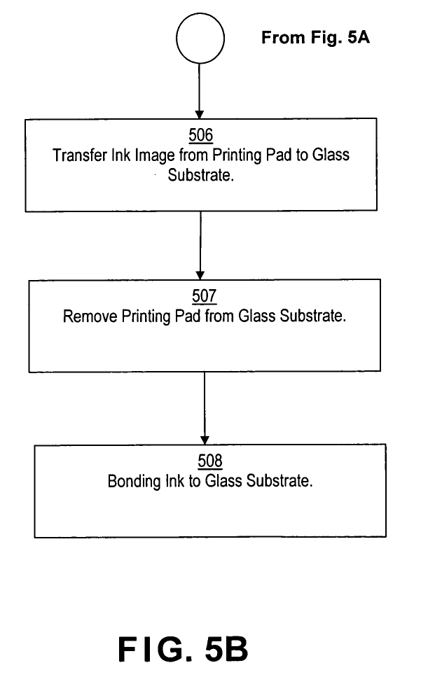

Turning now to FIG. 5, there is shown an embodiment of a method of utilizing a pad printer to pad print ink 120 of the instant invention on substrate 110. In step 501, substrate 110 is placed in a fixture of the pad printer. The fixture holds substrate 110 in a fixed position relative to the pad printing head. In certain embodiments, the fixture may utilize a vacuum and/or fixtures constructed to physically hold substrate 110 in place during the printing process. In multiple pad printing head machines, the fixture may move into various positions to enable each pad printing head in each pad printing station to apply ink 120 to substrate 110. In certain embodiments, the pad printer may comprise linear shuttle mechanisms, such as the fixture shown in A-66 through A-68 of Appendix A, or rotary indexing tables with multiple fixtures each containing a substrate 110. Such mechanisms index substrate 110 into position in each pad printing station for printing by each pad printing head.

In step 502, each pad printing head is adjusted and positioned relative to the fixture holding substrate 110 and that station's image plate. Positioning of substrate 110 may be made, for example, by shuttling or indexing the fixture bearing substrate 110 in the printing station so as to enable printing of ink 120 at the desired location of substrate 110 with the design etched in the image plate.

In step 503, ink 120 is applied to the image plate of the pad printing station. The image plate bears an etching of the pattern or design which is to be printed on substrate 110.

In step 504, the printing pad is put in contact with the image plate bearing ink 120 so as to transfer ink 120 from the image plate to the printing pad in the form of the design etched in the image plate.

In step 505, the printing pad is removed or lifted from the image plate bearing ink 120 in the design etched in the image plate.

In step 506, the printing pad bearing ink 120 in the design etched in the image plate is positioned relative to substrate 110 and put in contact with substrate 110 so as to transfer ink 120 from the printing pad to substrate 110 in the design etched in the image plate and in the desired location on substrate 110.

In step 507, the printing pad is removed or lifted from substrate 110 having transferred substantially all of ink 120 to substrate 110. Optionally, the printing process may then be repeated in the same print station to reprint the same design in substantially the same location on substrate 110 in order to, for example, increase opacity of printed ink 120. The fixture bearing substrate 110 may then be shuttled or indexed into position in another printing station in order to enable application of ink 120 in the same or another design in the same or another ink color. The printing process may then be repeated in an infinite number of designs and/or ink colors to achieve the complete design desired to be printed on substrate 110.

When employing the five station Printex printer described above, each station may comprise an image plate bearing a different image and/or an ink 120 to be printed on substrate 110. A substrate 110 may be mounted to each of the eighteen mounting fixtures and up to five substrates 110 may be printed simultaneously as each station may perform individual printing operations simultaneously. The Printex printer may be programmed to enable each printing station to perform an independent printing operation. For example, the first printing station may be programmed to print an image on a single substrate 110 three times at a particular depth, pressure and/or dwell times, while the second through fifth printing stations print their respective images two times at different depths, pressures or dwell times.

Once all print stations complete their respective printing sequences, the indexing table may index the mounting fixtures bearing substrates 110 to a different print station for additional printing. Once the printing of a substrate 110 has been completed, the fully printed substrate 110 may be removed from the fixture and replaced with an unprinted substrate 110 to be printed or moved directly to the next process step (e.g. Step 508), heating, firing and/or curing of ink 120 which bonds ink 120 to substrate 110. This cycling allows for continuous printing of substrates 110 and maximum utilization of the printer inasmuch as the printer may be kept in substantially continuous use.

In step 508, printed substrate 110 bearing ink 120 is heated in order to bond ink 120 to substrate 110. The heating may be carried out by any process which substantially permanently bonds ink 120 to substrate 110. Such processes may include, for example, firing or curing for times and at temperatures sufficient to bond ink 120 to substrate 110, which processes typically are dependent upon the compositions of inks 120 and substrate 110.

In one embodiment of the process of this invention, when employing the firing technique in the bonding process, printed substrate 110 is, for example, placed in a kiln at room temperature to a temperature required bond the vitreous enamel ink 120 to the substrate 110. For example, certain printed substrates 110 may be fired at a temperature of about 1225° F. to about 1250° F. for about 2 to about 3 minutes. Typically, in bonding step 508, printed substrate 110 is gradually brought up from room temperature to the peak firing temperature in order to avoid the fracturing of substrate 110 or to cause an adverse reaction or discoloration in ink 120.

In another embodiment of the instant invention, the method of FIG. 5 may be utilized to pad print an artificial deer eye or other design comprising a glass or glass-like substrate. In the deer eye embodiment, the printed design on the substrate may comprise the representation of the iris area which may be exclusive of the sclera portion of the artificial eye. The steps of the process of this embodiment comprise: (1) printing a dark blue pupil; (2) printing dark brown or black details which may include stippling in an iris, over-shading around pupil, shading at base of eye to create the appearance of a limbus band which is found at the perimeter of the iris (which may all be printed as one step or it may optionally be separated into two steps (stippling in one print and shading in another) if different colors are desired); (3) spraying background color; and (4) firing the printed substrate in a kiln at a temperature required bond the vitreous enamel to the substrate. For example, the printed substrate could be fired at a temperature of about 1225° F. to about 1225° F. for about 2 to about 3 minutes. Typically, in firing step 508, the substrate is gradually brought up to the peak firing temperature in order to avoid the fracturing of substrate 110 or to cause an adverse reaction or discoloration in ink 120.

An alternate method would be to print the background color and spray white as a secondary opacifying background. One reason to employ this method would be to use the combination of backgrounds to create a different tint. In this embodiment, the tint obtained is generally controlled by the mixture recipe of the vitreous enamel (viscosity and pigment load) and the number of times the image is repetitively printed on a single substrate. When using the pad printing device of this invention, repetitive printing on a single glass eye substrate in order to achieve the desired image on the substrate will be very consistent in the amount and precision by which the ink is applied, whereas a light, hand sprayed coating to achieve the same effect may vary depending on a number of variables which are difficult to control such as spraying equipment, relative humidity, human error, or the like.

Additionally, in the event that colorations of the inks as they appear on the substrate require adjustment during the printing process, the process may be halted while the color is adjusted and then restarted with limited or no waste of product. In other systems which utilize decals, the decals are typically ordered in large quantities weeks in advance of production. If the decals have defects it may take weeks, or longer, to obtain suitable replacements. Thus, significant amounts of product or valuable manufacturing time may be lost before any problem with the decals is noticed.

Moreover, if an artwork flaw is encountered while employing the vitreous enamels of this invention, or the vitreous enamel is improperly printed on the substrate, the vitreous enamel may usually be wiped off the substrate prior to the firing step, and the substrate may reused to print a new eye. In contrast, a decal may rip upon removal and is more expensive waste than the small amount of pigment lost to wiping off a product. It may also be more difficult to remove traditional inks from a substrate than the inks typically used in pad printing applications. In addition, it is difficult, and in some cases impossible, to effectively apply decalcomania transfers to concave surfaces such as, for example, the back of an artificial glass eye or other concave, convex or irregular surface.

The method of this invention is not labor intensive, is cost effective, is more consistent than the manual application of inks, and is far less prone to failure than decalcomania transfers or the application of other ink types.

In yet another embodiment of the invention, certain vitreous enamels may be successfully applied to metals, metal-like, and other substrates. For example, such vitreous enamels may be used to decorate copper, steel, and similar metals and their alloys. In addition, such vitreous enamels may also be used to decorate glass, ceramic and glass like substrates, as well as other substrates which tend to present surfaces which inks normally do not adhere to well. Thus, certain formulations of vitreous enamels may be developed to utilize the pad printing techniques of the instant invention to decorate metal and other substrates of various types such as jewelry, eating utensils, signage, kitchen and bathroom fixtures, automotive parts, as well as other applications. In one embodiment, certain vitreous enamels of this invention may be applied to copper alloy cuff links utilizing pad printing techniques of this invention. In yet another embodiment, certain vitreous enamels of this invention may be applied to kitchen and bathroom sink components utilizing pad printing techniques of this invention.

In one embodiment of the vitreous enamels of this invention which are suitable for application to metal and other substrates, particularly where a non-lead based ink is desired, a substantially balanced mixture of a zinc base flux and a bismuth base flux are utilized in the vitreous enamels. It should be noted, however, that other proportions of bismuth and zinc fluxes may be used depending on the substrate to which the enamel is to be adhered, as well as the other characteristics of the enamel which may be desired. For example, such an ink may comprise a non-lead based flux, such as bismuth and/or zinc; a clear vinyl based ink; and a thinner, wherein said ink is able to be utilized in the printing of images on or otherwise decorating a substrate.

Although typically expensive, the bismuth flux comprises a very hard glass and provides excellent wear resistance characteristics to the enamel. On the other hand, zinc flux is typically less expensive, but also less wear resistant than that of the more expensive bismuth flux. However, the color palette available for zinc fluxes is larger than that of bismuth fluxes. Accordingly, combining the bismuth and zinc fluxes in appropriate proportions will yield a vitreous enamel which provides an improved wear resistant and less expensive enamel while providing a wider array of color choices than if zinc flux was used alone. Such enamels may be used as specific opaque or transparent colors or as four color process colors depending upon the pigments used and the percentage of pigment in comparison to flux and medium used. The disclosure herein is directed to the variations and modifications of the elements and methods of the invention disclosed that will be apparent to those skilled in the art in light of the disclosure herein. Thus, it is intended that the present invention covers the modifications and variations of this invention, provided those modifications and variations come within the scope of the appended claims and the equivalents thereof.

What is claimed is:

1. An ink comprising:
a vitreous enamel in dry powder form;
a clear vinyl based ink; and
a thinner, wherein said ink is able to be utilized in the printing of images on a glass, glass-like, or ceramic substrate.

2. A method for applying ink to a glass, glass-like, or ceramic substrate comprising the steps of:
printing an ink on said substrate, wherein said ink comprises a vitreous enamel in dry powder form; a clear vinyl based ink; and a thinner; and
heating said printed substrate to a temperature which substantially bonds said ink to said substrate.

3. An artificial eye comprising:
a glass, glass-like, or ceramic substrate having substantially concave and convex surfaces;
a layer of decorative ink applied to said concave surface of said substrate, wherein said decorative ink comprises a vitreous enamel in dry powder form; a clear vinyl based ink base; and a thinner.

4. The artificial eye of claim 3, wherein said substrate substantially comprises crystal.

5. The artificial eye of claim 3, wherein said decorative ink is applied by pad printing to said concave surface of said substrate.

6. The artificial eye of claim 3, wherein said decorative ink layer is in a form which substantially resembles one or more components an eye.

7. The artificial eye of claim 3, wherein said artificial eye substantially resembles one or more components an eye of an animal.

8. The artificial eye of claim 3, further comprising a layer of background ink applied between said decorative ink and barrier layers.

9. The artificial eye of claim 3, wherein said barrier layer comprises a rubberized coating.

10. The artificial eye of claim 3, wherein said barrier layer comprises automotive undercoating.

11. The artificial eye of claim 3, wherein said artificial eye is mounted to a form used in taxidermy.

12. A method for producing an artificial eye, said eye comprising a glass or glass like substrate, said substrate having substantially concave and convex surfaces, wherein said method comprises the steps of:
applying a layer of decorative ink to said concave surface of said substrate, wherein said decorative ink layer comprises a vitreous enamel in dry powder form; a clear vinyl based ink base; and a thinner; and
heating said printed substrate to a temperature which substantially bonds said ink to said substrate.

13. The method of claim 12, wherein said substrate substantially comprises crystal.

14. The method of claim 12, wherein said decorative ink is applied by pad printing to said concave surface of said substrate.

15. The method of claim 12, wherein said decorative ink layer is in a form which substantially resembles one or more components of an eye.

16. The method of claim 12, wherein said artificial eye substantially resembles one or more components of an eye of an animal.

17. The method of claim 12, further comprising the step of applying a layer of background ink to said decorative ink layer.

18. The method of claim 12, wherein said eye is mounted to a form used in taxidermy.

19. A method for printing inks on the surface of an artificial eye, comprising the steps of:
transferring said ink in the form of an image from an image plate to a printing pad, wherein said ink comprises a vitreous enamel in dry powder form; a clear vinyl based ink base; and a thinner; and
transferring said ink image from said printing pad to said surface of said eye.

20. A method for printing inks on glass or glass like surface comprising the steps of:
transferring said ink in the form of an image from an image plate to a printing pad, wherein said ink comprises a vitreous enamel in dry powder form; a clear vinyl based ink base; and a thinner; and
transferring said ink image from said printing pad to said surface of said glass or glass-like surface.

21. An ink comprising:
a non-lead based flux;
a clear vinyl based ink; and
a thinner, wherein said ink is able to be utilized in the printing of images on a substrate.

22. The ink of claim 21 wherein said non-lead flux comprises one or more of bismuth flux and zinc flux.

23. The ink of claim 21 wherein said non-lead flux comprises one or more of bismuth flux and zinc flux.

24. The ink of claim 21 wherein said substrate is a metal or metal-like substrate.
